(19) United States
(12) Patent Application Publication
(10) Pub. No.: US 2013/0337525 A1
(43) Pub. Date: Dec. 19, 2013

(54) Method of Producing Ethanol

(71) Applicants: Tomoki Hayakawa, Tokyo (JP); Masanori Sato, Tokyo (JP)

(72) Inventors: Tomoki Hayakawa, Tokyo (JP); Masanori Sato, Tokyo (JP)

(73) Assignee: TSUKISHIMA KIKAI CO., LTD., Tokyo (JP)

(21) Appl. No.: 14/001,370

(22) PCT Filed: Dec. 13, 2011
(86) PCT No.: PCT/JP2011/078821

§ 371 Date: Aug. 23, 2013

(30) Foreign Application Priority Data
Feb. 24, 2011 (JP) 2011-038338

(51) Int. Cl. C12P 7/10

(52) U.S. Cl. CPC C12P 7/10
USPC 435/165

(57) ABSTRACT

A method of producing ethanol by adding a cellulosic raw material treatment liquid to a sugar-containing liquid and then performing ethanol fermentation, in which the sugar-containing liquid is one or more types selected from the group consisting of extracted juice of crops, molasses, and enzyme-treated products of cereals containing a water-soluble saccharide, and the cellulosic raw material treatment liquid is either a sugar solution derived from cellulosic raw materials obtained by saccharification of the cellulosic raw materials or a fermentation broth derived from the cellulosic raw materials obtained by ethanol fermentation of the sugar solution derived from the cellulosic raw materials.

### BACKGROUND OF THE INVENTION

1. Field of the Invention

The present invention relates to a method of producing high concentration ethanol with high efficiency from cellulosic raw materials and sugar-containing liquid.

Priority is claimed on Japanese Patent Application No. 2011-038338, filed Feb. 24, 2011, the content of which is incorporated herein by reference.

2. Description of Related Art

In recent years, as measures to curb global warming, attempts to produce ethanol with high efficiency from botanical biomass and use the ethanol for fuel or as a chemical raw material have been promoted worldwide. The production process which produces ethanol from cellulosic biomass has been attracting attention because it doesn't affect the food supply chain unlike other botanical biomass.

As a method of producing ethanol from the cellulosic biomass, it is general that cellulosic biomass is pretreated prior to saccharification with enzyme, then followed by ethanol fermentation (refer to Patent Documents 1 and 2). On the other hand, ethanol has conventionally been produced from sugar-containing liquid, like as ethanol production in a sugar factory from molasses.

In recent years, with the improvements in ethanol production technology from cellulosic biomass as described above, a system which combines ethanol production from cellulosic biomass and ethanol production from sugar-containing liquid has been proposed. For example, in Non-Patent Documents 1 and 2, methods of producing ethanol are disclosed which combine and distill ethanol-containing fermentation broth derived from molasses, which is a sugar-containing liquid, and ethanol-containing fermentation broth derived from bagasse (the residue after crushing sugar cane), which is a cellulosic biomass as shown in FIG. 1.

### PATENT DOCUMENTS

[Patent Document 1] Japanese Unexamined Patent Application, First Publication No. 2005-168335
[Patent Document 2] Japanese Unexamined Patent Application, First Publication No. 2009-022165

### NON-PATENT DOCUMENTS

[Non Patent Document 1] “Fiscal Year 2004 Research Report: Molasses And Bagasse Ethanol Producing Model Project Feasibility Study In Sugar Factories” New Energy and Industrial Technology Development Organization Review
[Non Patent Document 2] “Operational Report from That Roong Ruang Energy Co., Ltd., Ugrit Asadatorn” Thailand Sugar Industrial Development Bureau homepage: http://oldweb.ocsb.go.th/uploads%5Ccontents%5C4%5Cattachfiles%5CTRE%20Presentation%204-06-09-1.pdf

### DISCLOSURE OF INVENTION

### Problems to be Solved by the Invention

During enzymatic saccharification with cellulosic biomass as a raw material as described above, in a case where the insoluble solid concentration in the saccharification tank is too high, the saccharification efficiency is low. Accordingly, the sugar yield after saccharification decreases. Therefore, in general, a diluting water is introduced into the saccharification tank before saccharification process and the insoluble solid concentration in the saccharification tank is adjusted to the proper range. However, in such a case, there is a problem in that large amount of diluting water is necessary.

On the other hand, during the ethanol fermentation with a sugar-containing liquid as a raw material, in a case where the prepared sugar concentration is too high, the ethanol concentration in the fermentation tank rise above the appropriate level. Accordingly, since the high concentration ethanol inhibits the ethanol fermentation itself due to yeast in the fermentation tank, the ethanol yield after fermentation is reduced. For example, in a case where the sugar-containing liquid is molasses having approximately 50 mass % of sugar in general, the ethanol concentration in the fermentation tank in the fermentation treatment is extremely high with a sugar concentration of 50 mass %. Therefore, dilution to adjust the concentration of sugar in the fermentation tank is generally conducted by inducing diluting water, in case of using some kind of sugar liquid as raw material. However, in such a case, there is a problem in that large amount of diluting water is necessary.

Furthermore, in a case where the diluting water is introduced before the above-described saccharification process and before the fermentation treatment, the ethanol concentration in the obtained fermentation broth is reduced. Therefore, it causes a problem that the energy consumption required for producing high concentration ethanol through the distillation process increases. Furthermore, there is also a problem in that the amount of waste water which is discharged from the ethanol producing is increased.

In addition, in the method of producing ethanol described in Non-Patent Documents 1 and 2, fermentation broth derived from molasses and a fermentation broth derived from bagasse is mixed then distilled in the same process. At this time, the saccharification followed by fermentation from cellulosic biomass and the fermentation from the sugar-containing liquid are respectively performed in separate tanks. Therefore, it is necessary to add the diluting water with respect to each tank, and there is a problem in that large amount of diluting water is necessary.

The fermentation of the sugar-containing liquid is performed by adding the diluting water so as not to exceed the ethanol tolerance concentration of the microorganisms used in the fermentation. On the other hand, in the case of saccharification and fermentation of cellulose, the diluting water is added for the purpose of performing saccharification with high efficiency by adjusting the solid concentration, as described above. Accordingly, there is a problem in that the ethanol in the cellulose saccharification and fermentation broth has a concentration which is lower than the ethanol tolerance concentration of the microorganisms used in the fermentation and lower than the general ethanol concentration of the sugar-containing liquid fermentation broth.

At present, in the distillation step, a solution where both fermentation broths are mixed is supplied; however, as a result, the ethanol concentration in the mixed liquids is substantially lower than the tolerance concentration of the microorganisms.

The present invention has been made in view of the above circumstances and provides a method of producing ethanol which is able to produce high concentration ethanol with high efficiency from cellulosic raw materials and sugar-containing liquid.

### Means to Solve the Problems

As a result of intensive studies to solve the above-described problem, the inventors found that it is possible to produce high concentration ethanol with high efficiency from a cellulosic raw material and a sugar-containing liquid by using a mixture obtained by adding either one of a sugar solution derived from cellulosic raw materials obtained by saccharification of the cellulosic raw materials or a fermentation broth derived from cellulosic raw materials obtained by ethanol fermentation of the sugar solution derived from the cellulosic raw materials during the ethanol fermentation of the sugar-containing liquid, thereby completing the present invention.

That is, the present invention provides the following (1) to (6).

(1) A method of producing ethanol which produces ethanol by adding a liquid generated from processing cellulosic raw material to a sugar-containing liquid and then performing ethanol fermentation thereon, in which the sugar-containing liquid is one or more types selected from the group consisting of extracted juice of crops, molasses, and enzyme-treated products of cereals containing a water-soluble saccharide, and the cellulosic raw material treatment liquid is either a sugar solution derived from cellulosic raw materials obtained by saccharification of the cellulosic raw materials or a fermentation broth derived from the cellulosic raw materials obtained by ethanol fermentation of the sugar solution derived from the cellulosic raw materials.

(2) The method of producing ethanol according to (1) in which the sugar concentration in a fermentation tank for ethanol fermentation of the sugar-containing liquid is 25 mass % or less.

(3) The method of producing ethanol according to (1) or (2) in which the ethanol concentration obtained after the ethanol fermentation of the sugar-containing liquid is 12 mass % or less.

(4) The method of producing ethanol according to any one of (1) to (3) in which a saccharifying step and a fermenting step of the cellulosic raw materials are performed in the same reaction tank.

(5) The method of producing ethanol according to any one of (1) to (4) in which the cellulosic raw materials are one or more types selected from the group consisting of bagasse, rice straw, wheat straw, chaff, wheat shells, cassava residue, and corn stover, and the enzyme-treated product of the cereal is a starch saccharification liquid obtained by saccharification of one or more types selected from the group consisting of rice, wheat, cassava, and corn.

(6) The method of producing ethanol according to any one of (1) to (5) in which the cellulosic raw material is bagasse and the sugar-containing liquid is extracted juice of crops containing a water-soluble saccharide or molasses.

### Effect of the Invention

In the method of producing ethanol of the present invention, cellulosic raw material treatment liquid is used in place of the conventional water as a part of the diluting water during the ethanol fermentation of the sugar-containing liquid. Accordingly, it is possible to reduce the consumption of diluting water used compared to a case where ethanol fermentation from the cellulosic raw materials and ethanol fermentation of the sugar-containing liquid are separately performed. As a result, it is possible to achieve a reduction in production costs and an improvement in production efficiency, and it is possible to obtain high concentration ethanol after fermentation.

In addition, according to the method of producing ethanol of the present invention, the ethanol in the obtained fermentation broth has a high concentration. Accordingly, it is possible to reduce the energy required at the time of distilling the ethanol fermentation broth.

### BRIEF DESCRIPTION OF DRAWINGS

FIG. 1 is a flow chart showing a conventional method of producing ethanol using bagasse and molasses.

FIG. 2 is a view showing a calculation method for determining the amount of sugar-containing liquid, the sugar solution derived from cellulosic raw materials, and the diluting water in a case where the ethanol target concentration after fermentation (the microorganism ethanol tolerance upper limit concentration) is 12%.

FIG. 3 is a flowchart showing a control concept for determining the amount of sugar-containing liquid, the sugar solution derived from cellulosic raw materials, and the diluting water in a case where the ethanol target concentration after fermentation (the microorganism ethanol tolerance upper limit concentration) is 12%.

FIG. 4 is a view showing a calculation method for determining the amount of sugar-containing liquid, the fermentation broth derived from cellulosic raw materials, and the diluting water in a case where the ethanol target concentration after fermentation (the microorganism ethanol tolerance upper limit concentration) is 12%.

FIG. 5 is a flowchart showing a control concept for determining the amount of sugar-containing liquid, the fermentation broth derived from cellulosic raw materials, and the diluting water in a case where the ethanol target concentration after fermentation (the microorganism ethanol tolerance upper limit concentration) is 12%.

FIG. 6 is a flowchart showing the production method of the present invention using a sugar solution derived from bagasse and molasses (sugar-containing liquid) in a case where the ethanol target concentration after fermentation (the microorganism ethanol tolerance upper limit concentration) is 10%.

FIG. 7 is a flowchart showing the production method of the present invention using a fermentation broth derived from bagasse and molasses (sugar-containing liquid) in a case where the ethanol target concentration after fermentation (the microorganism ethanol tolerance upper limit concentration) is 10%.

### EMBODIMENTS FOR CARRYING OUT THE INVENTION

The method of producing ethanol of the present invention produces ethanol by adding a cellulosic raw material treatment liquid to a sugar-containing liquid and then performing ethanol fermentation thereon.

### (Sugar-Containing Liquid)

In the present invention, the sugar-containing liquid is one or more types selected from the group consisting of the extracted juice of crops, molasses, and an enzyme-treated product of cereal containing a water-soluble saccharide including monosaccharides such as glucose, galactose, mannose, fructose, sorbose, allose, talose, gulose, altrose, idose, xylose, arabinose, ribose, and lyxose, or oligosaccharidessuch as sucrose, trehalose, lactose, maltose, cellobiose, raffinose, and cellotriose where the above monosaccharides are the unit component.

Examples of crops containing a water-soluble saccharide include cane juice, beet juice, sorghum juice, or the like.

Examples of the cereals include rice, wheat, cassava, corn, sorghum, foxtail millet, Japanese millet, or the like.

The method for enzymatic treatment of the cereals is not particularly limited and it is possible to perform the treatment by bringing an enzyme such as amylase into contact with the cereals. In the present invention, from the viewpoint of a high starch content and large production volume, it is preferable to use a starch saccharification liquid obtained by saccharification of one or more types selected from the group consisting of rice, wheat, cassava, and corn.

The extracted juice of crops, molasses, and an enzyme-treated product of cereal containing a water-soluble saccharide may be used as is, or may be used after performing a desalination treatment or a sterilization treatment. The methods of the desalination treatment and the sterilization treatment are not particularly limited, and it is possible to use any known method.

In the present invention, the sugar-containing liquid is preferably a liquid which contains 5 mass % or more of sugar, more preferably a liquid which contains 10 mass % or more of sugar, even more preferably a liquid which contains 15 mass % or more of sugar, and particularly preferably a liquid which contains 20 mass % or more of sugar.

### (Cellulosic Raw Material Treatment Liquid)

In the present invention, the cellulosic raw material treatment liquid is any one of a sugar solution derived from cellulosic raw materials obtained by saccharification of the cellulosic raw materials or a fermentation broth derived from cellulosic raw materials obtained by ethanol fermentation of the sugar solution derived from the cellulosic raw materials.

The cellulosic raw materials are not particularly limited as long as cellulose is contained therein and may be a herbaceous biomass, a wood-based biomass, or another cellulosic biomass. The cellulosic raw materials may be cultivated crops such as rice, wheat, corn, sugar cane, sugar beet, hemp, cotton, sorghum, Erianthus, or cassava. In particular, the cellulosic materials which are to be disposed and include high content of cellulose are preferable (for this purpose). Specifically, examples of the cellulosic raw materials include agricultural residues such as rice straw, wheat straw, chaff, wheat shells, corn stover, bagasse, coconut husks, and cassava residue (residue of cassava after starch recovery), forestry residues such as bamboo, wood chips, and timber from forest thinning, waste paper, old clothes, or the like. Among the above-described raw materials, soft cellulosic biomass such as herbaceous biomass is preferable as the cellulosic raw material. In addition, from the viewpoint of being available in large quantities at low cost, cereal residues such as bagasse, rice straw, wheat straw, chaff, wheat shells, cassava residue, or corn stover are preferable as raw material. In the present invention, among the cellulosic raw materials described above, only one type of raw material may be used, or two or more types of raw material may be used.

As the cellulosic raw material in the present invention, in particular, raw materials obtained in the same field of industry as the sugar-containing liquid to be used are preferable, and, as the above-described sugar-containing liquid, those obtained from the same food factory or the like are even more preferable. In a case where it is possible to obtain the cellulosic raw materials and the sugar-containing liquid from the same factory, farm, or the like, ethanol production by the method of the present invention can be carried out in a plant which is built near the factory or the like. As a result, it is possible to reduce the cost of transporting the cellulosic raw material or the sugar-containing liquid.

Specifically, a case where bagasse and molasses, which are obtained in a sugar factory, are used as the cellulosic raw material and as the sugar-containing liquid respectively; a case where rice straw or chaff, which are obtained in the field of rice plant or rice processing factories, is used as the cellulosic raw material and enzyme-treated products of rice which has been discarded are used as the sugar-containing liquid; or a case where wheat straw or wheat shells, which are obtained in a wheat processing factory are used as the cellulosic raw material and enzyme-treated products of wheat which has been discarded are used as the sugar-containing liquid, are preferable. In addition, a case where starch production residue (cassava residue) obtained in a cassava starch factory is used as the cellulose raw material content and enzyme-treated products of cassava which have been discarded are used as the sugar-containing liquid; a case where corn stover is used as the cellulosic raw material and enzyme-treated products of corn which have been discarded are used as the sugar-containing liquid, both of which are obtained in a corn factory; or a case where bagasse is used as the cellulosic raw material and extracted juice (cane juice) from squeezing sugar cane which has been discarded is used as the sugar-containing liquid, both of which are obtained in a sugar factory, are preferable. In addition, a case where sugar beet residue obtained in a sugar factory is used as the cellulosic raw material and extracted juice (beet juice) from squeezing sugar beet (sugar beet) which have been discarded is used as the sugar-containing liquid; or a case where sorghum residue obtained in a sugar factory is used as the cellulosic raw material and extracted juice (sorghum juice) from squeezing millet which has been discarded is used as the sugar-containing liquid, are preferable.

In particular, a case where bagasse and the extracted juice of crops or molasses are used is preferable since a step of enzyme treating cereals is not necessary.

In addition, in the present invention, the cellulosic raw materials are preferably pre-treated according to a pre-treatment step.

The method of pre-treating the cellulosic raw materials is not particularly limited as long as it is a method where it is possible to improve the saccharification efficiency in the subsequent saccharifying steps by being performed with respect to the cellulosic raw materials. Preferably, as the pre-treatment method, for example, one or two or more methods selected from treatment methods formed of a cutting treatment and a hydrothermal treatment may be combined and used.

The method of the cutting treatment is not particularly limited and it is possible to perform the method using a known apparatus or the like; however, for example, it is possible to cut the cellulosic raw materials to a size of 1 to 20 mm, more preferably a size of 1 to 5 mm according to the cutting treatment.

The method of the hydrothermal treatment is not particularly limited and it is possible to perform the method using a known apparatus or the like.

The conditions of the hydrothermal treatment are not particularly limited. However, the hydrothermal treatment is preferably performed in saturated steam at 160 to 250° C., more preferably performed in saturated steam at 170 to 220° C. In addition, specifically, the hydrothermal treatment is preferably performed under pressure conditions of 0.7 to 2.3 MPaG. In addition, the hydrothermal treatment is preferably performed within a residence time of 3 to 120 minutes, more preferably 5 to 30 minutes. By performing the hydrothermal treatment under the preferable conditions described above, it is possible to further reduce excessive degradation of the cellulosic raw materials.

In addition, in place of the above-described cutting treatment and the hydrothermal treatment or in combination with these treatments, it is possible to perform known pre-treatments such as a chopping treatment, a fine milling treatment, an alkali treatment, a bacterial treatment, a sulfuric acid treatment, a thermal softening treatment or a solvolysis treatment.

In addition, in place of the above-described treatments or in addition to the above-described treatments, a defibration treatment is also preferably performed. The method of the defibration treatment is not particularly limited. For example, the defibration treatment is performed using a pulp disintegrator normally used in the paper industry. More specifically, the defibration treatment is performed using a low concentration (solid concentration of less than 6 mass %) pulper, a medium concentration (solid concentration of 6 to 10 mass %) pulper, a high concentration (solid concentration of 10 to 30 mass %) pulper, or the like. The defibration treatment is more preferably performed additionally after the hydrothermal treatment.

In addition, the liquid ratio in the cellulosic raw materials treated by the methods described above may be prepared using a known separator such as a centrifugal separator or a belt concentrator as necessary.

Sugar Solution Derived from Cellulosic Raw Material

The sugar solution derived from the cellulosic raw materials in the present invention is obtained by saccharification of cellulosic raw materials or pre-treated cellulosic raw materials (below, may be referred to as “pre-treated product”) subjected to the above-described pre-treatments.

The method of performing the saccharification is not particularly limited and it is possible to perform the method using a conventionally known method. For example, by performing an enzyme treatment by adding a saccharification enzyme such as cellulase on the cellulosic raw materials or the pre-treated products obtained by the above-described method, it is possible to obtain a sugar solution where cellulose or hemicellulose is saccharified. Known conventional enzymes may be used as the saccharification enzyme, including ones derived from Trichoderma reesei or ones derived from Acremonium cellulolyticus. In a case where the insoluble solid concentration of the cellulosic raw material used in the saccharification is too high, as described above, the saccharification efficiency is lowered. To prevent the decrease in the saccharification efficiency, the insoluble solid concentration is preferably 25 mass % or less. The method of setting the insoluble solid concentration of the cellulosic raw material to the above-described range is not particularly limited; however, for example, it is possible to add a diluting water to the cellulosic raw materials or pre-treated products as necessary. The use of water as the diluting water is preferable.

In addition, the saccharification of the cellulosic raw materials or the pre-treated products may be performed a batch process where the whole raw material is processed at the same time, or multiple stage process where the saccharification of raw material is performed in a stepwise way. Since the cellulosic raw materials or the pre-treated products of the initial saccharification stage have a high solid concentration, relatively high agitation power in saccharification tank is required only in the initial saccharification stage. Therefore, only an early period, for example, initial 1 to 5 hours, of saccharification is performed in a tank with a high agitating power. Thereafter, the saccharification of the remaining cellulosic raw materials or the pre-treated products is preferably performed in a separate tank. That is, by performing saccharification in multiple stages, it is possible to perform the saccharification efficiently.

In addition, in place of the method of adding the total amount of cellulosic raw material only in the initial stage of the saccharification, it is possible to use a fed-batch manner where the raw materials are added gradually along with the progress of the saccharification. In a case of using this method, the solid concentration in the tank is low in the initial period and additional solid is added along with the decrease of solid concentration decreases due to the progress of the saccharification. As a result, there is an advantage in that the solid concentration in the tank is kept low at all times.

Fermentation Broth Derived from Cellulosic Raw Materials

The fermentation broth derived from cellulosic raw materials in the present invention is obtained by the ethanol fermentation of the sugar solution derived from the cellulosic raw materials.

The method of subjecting the sugar solution derived from the cellulosic raw materials to ethanol fermentation is not particularly limited. It is possible to use a method normally used in the ethanol fermentation of sugar solutions. For example, by using a method where known microorganisms having the ability of ethanol fermentation are added to the sugar solution derived from the cellulosic raw materials in the ethanol fermentation tank, the fermentation broth derived from cellulosic raw materials is obtained.

As the microorganisms having the ability of ethanol fermentation, for example, yeast of the Saccharomyces genus, specifically, Saccharomyces cerevisiae, and the like may be exemplified.

When the sugar solution derived from the cellulosic raw materials is subjected to ethanol fermentation, in a case where the sugar concentration in the ethanol fermentation tank is excessively high, the ethanol concentration in the fermentation tank during the fermentation treatment becomes higher than the appropriate value, as described above. Therefore, ethanol fermentation by the yeast in the fermentation tank is inhibited by the ethanol. As a result, the ethanol yield per unit amount of sugar is reduced. In order to prevent the reduction in the ethanol yield per unit amount of sugar, the sugar concentration in the ethanol fermentation tank is preferably 25 mass % or less, more preferably 22 mass % or less, and most preferably 20 mass % or less.

The method of adjusting the sugar concentration in the ethanol fermentation tank to the above-described ranges is not particularly limited, and it is possible to use a method of adding a diluting water, or the like. Water is preferable as the diluting water.

Here, residual sugars may be contained in trace amounts in the fermentation broth derived from the cellulosic raw materials in the present invention.

In recent years, from the points of view of simplifying the producing process and improving the efficiency, methods of performing saccharification and fermentation at the same time have also been used. The saccharification process and the ethanol fermentation treatment of the above-described cellulosic raw materials may be performed independently in separate tanks, or may be performed at the same time in the same tank.

### (Ethanol Fermentation of Sugar-Containing Liquid)

The amount of cellulosic raw material treatment liquid to be added to the sugar-containing liquid before the ethanol fermentation is not particularly limited; however, the added amount is preferably determined such that the ethanol concentration in the fermentation broth is 12 mass % or less. In addition, the added amount is more preferably determined such that the ethanol concentration in the fermentation broth is 10 mass % or less. In addition, in a case where only the cellulosic raw material treatment liquid is added and the above-described ethanol concentration is not satisfied, it is possible to further add a diluting water.

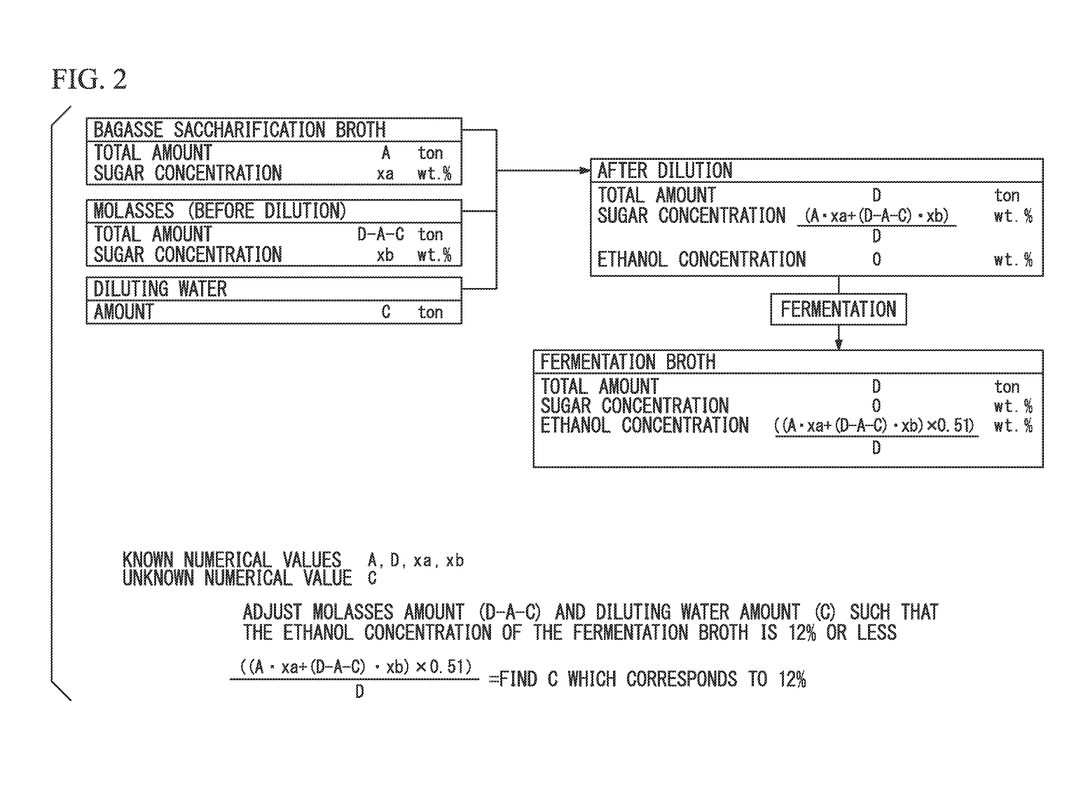

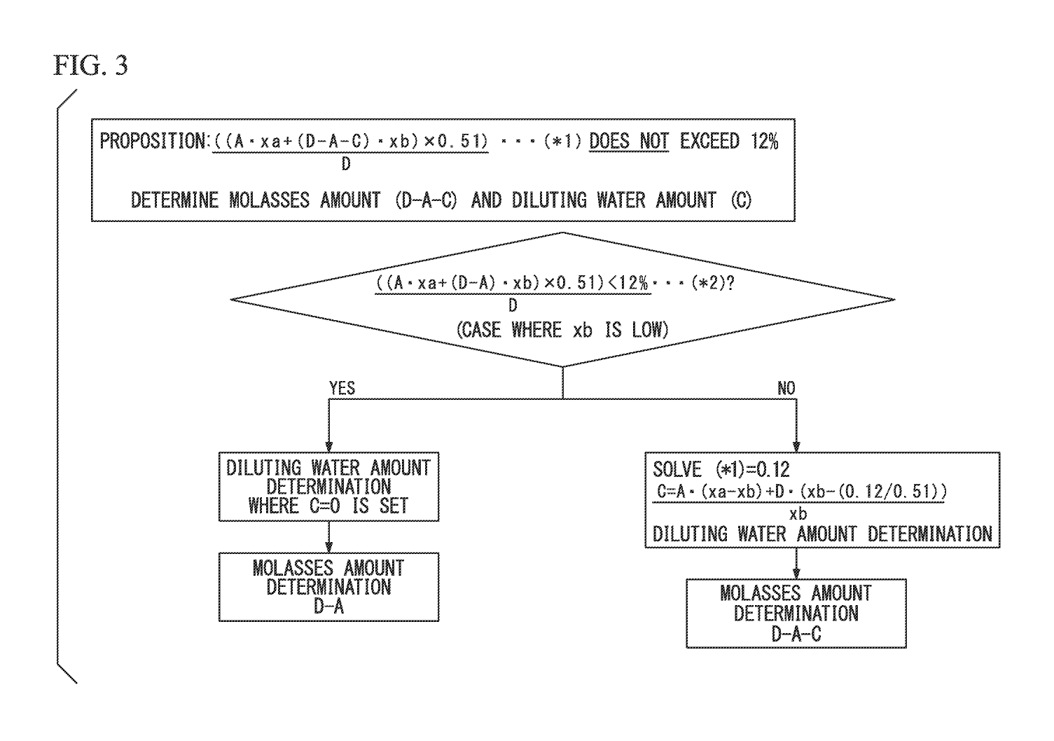

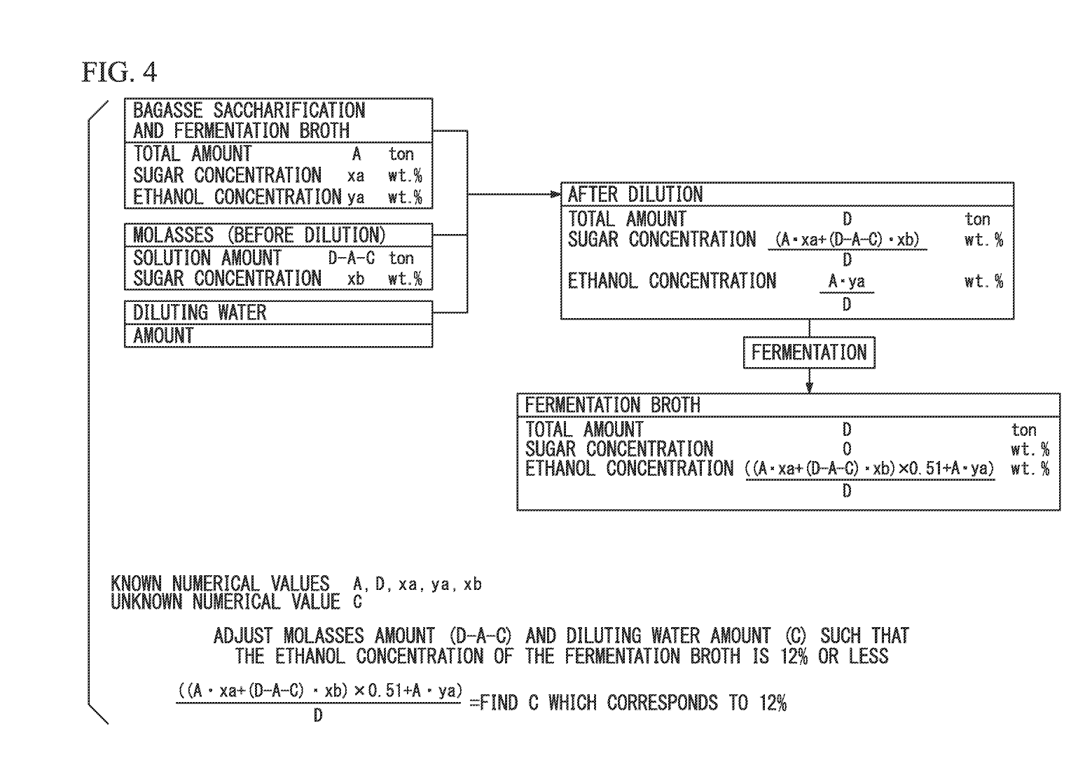

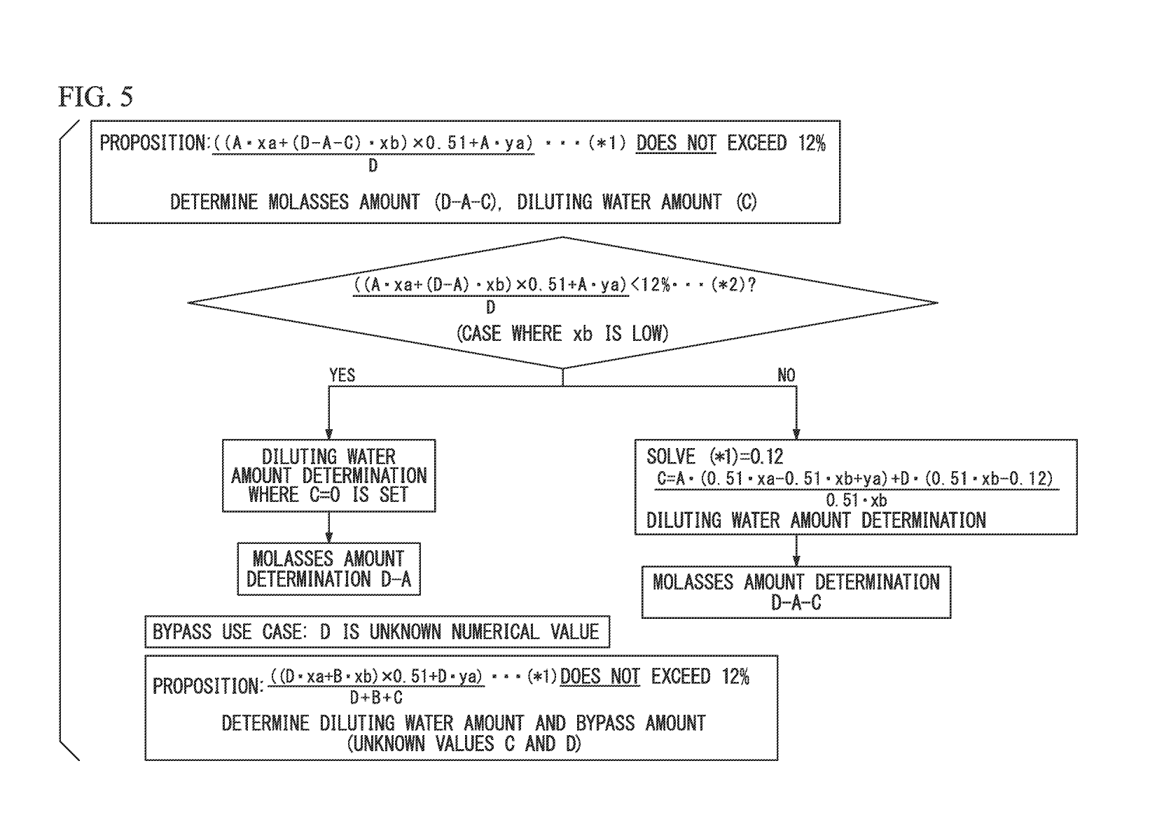

Specifically, it is possible for the amounts of the sugar-containing liquid and the sugar solution derived from cellulosic raw materials where the ethanol concentration in the fermentation broth is 12 mass % or less to be determined according to the calculation method which is shown in FIG. 2 and the control concept which is shown in the flowchart of FIG. 3, for example. In addition, it is possible for the amounts of the sugar-containing liquid and the fermentation broth derived from cellulosic raw materials where the ethanol concentration in the fermentation broth is 12 mass % or less to be determined according to the calculation method which is shown in FIG. 4 and the control concept which is shown in the flowchart of FIG. 5, for example.

In FIG. 2 to FIG. 5, A indicates the amount of liquid generated from processing cellulosic raw materials (sugar solution in FIG. 2 and FIG. 3, and fermentation broth in FIG. 4 and FIG. 5), xa indicates the sugar concentration in the cellulose raw materials treatment liquid, ya indicates the ethanol concentration in the cellulose raw materials treatment liquid, D indicates the total amount in the fermentation tank, xb indicates the sugar concentration in the sugar-containing liquid, and C indicates the amount of diluting water to be added. Here, in FIG. 2 to FIG. 5, the theoretical yield from sugar to ethanol is set to 51 mass %.

In the present invention, the method of subjecting the sugar-containing liquid and the cellulosic raw materials treatment liquid to ethanol fermentation is not particularly limited and it is possible to use a method which is normally used in the ethanol fermentation of sugar solutions. For example, by using a method of adding known microorganisms having the ability of ethanol fermentation to the sugar solution and causing ethanol fermentation of the sugar solution, it is possible to obtain a fermentation broth which includes ethanol. As the microorganisms having the ability of ethanol fermentation, the same ones as mentioned above may be exemplified. Here, in a case where the fermentation broth derived from cellulosic raw materials is added to the sugar-containing liquid for dilution, there is an effect where contamination in the sugar-containing liquid fermenting step is suppressed by the contained ethanol.

The fermentation broth including the ethanol which is obtained by the above-described ethanol fermentation is usually made into a commercial product through the steps of distillation, purification, and the like. The fermentation broth which is obtained by the method of producing ethanol of the present invention has a high ethanol concentration. Therefore, it is possible to reduce the energy required in the step of distillation, and it is possible to obtain high purity ethanol with better efficiency.

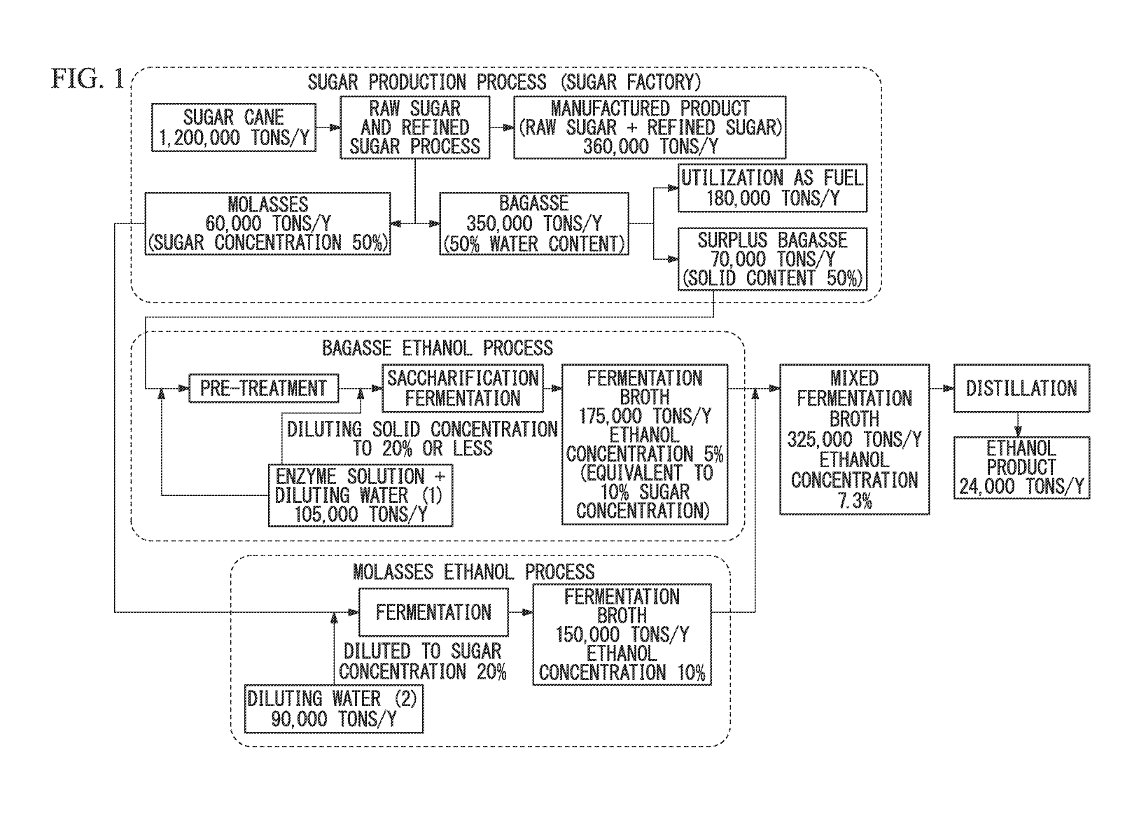

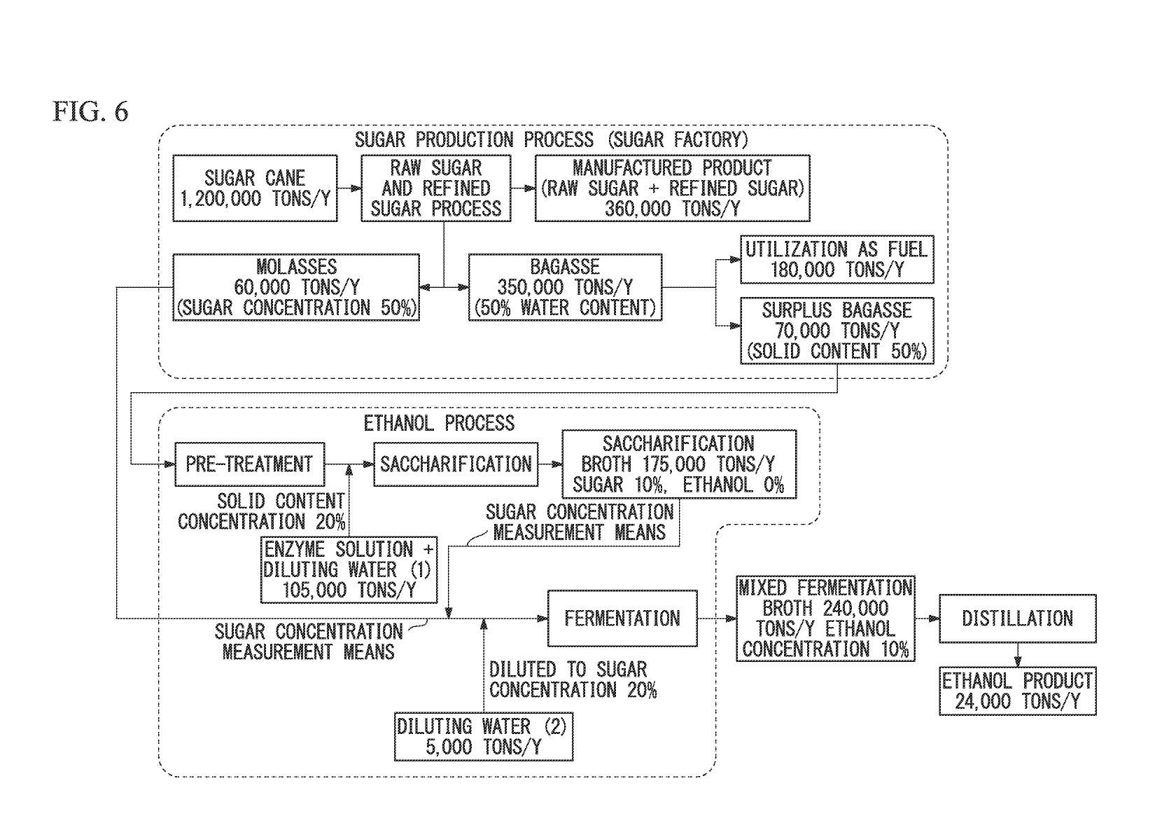

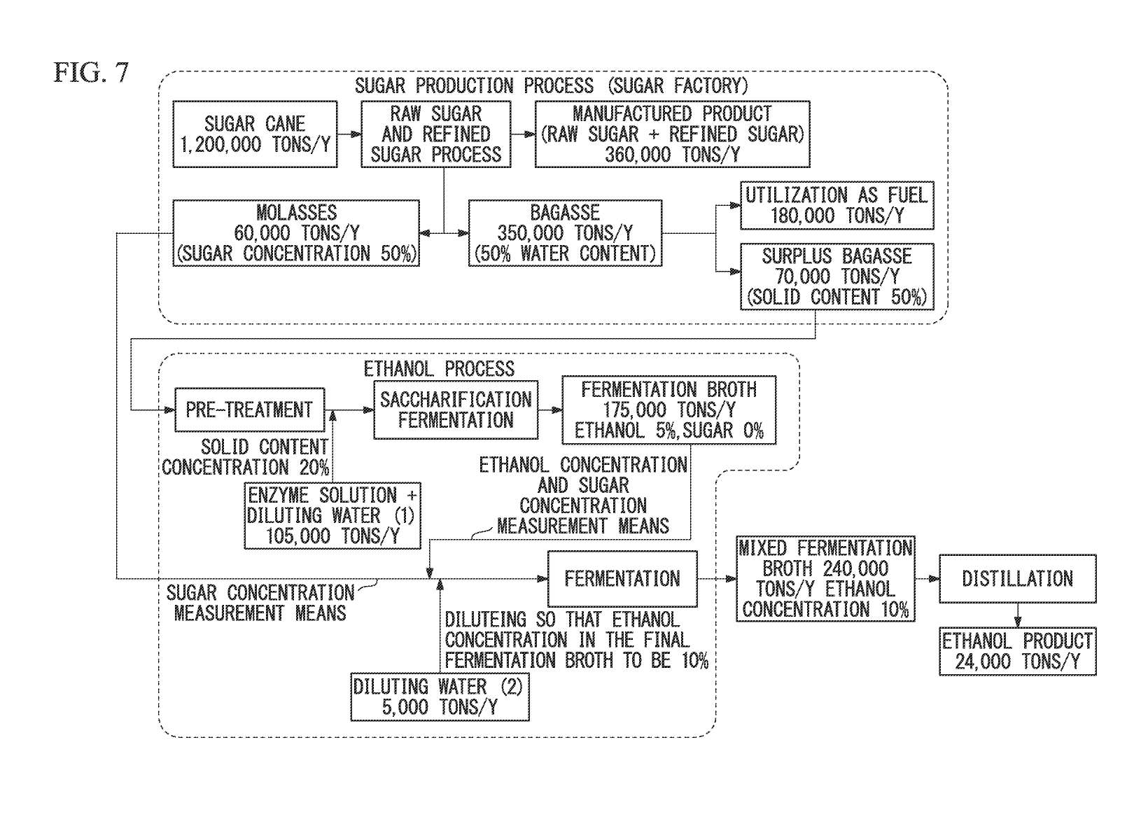

FIG. 6 and FIG. 7 show specific examples of the method of producing ethanol of the present invention. FIG. 6 shows a specific example where a sugar solution derived from cellulosic raw materials is used as the cellulosic raw material treatment liquid. FIG. 7 shows a specific example where a fermentation broth derived from cellulosic raw materials is used as the cellulosic raw material treatment liquid. Here, the amounts of molasses and bagasse which are used in FIG. 6 and FIG. 7 are the same as in the conventional method of producing ethanol of FIG. 1. In addition, the ethanol target concentration after fermentation (the microorganism ethanol tolerance upper limit concentration) in each of FIG. 1, FIG. 6, and FIG. 7 is 10%.

Comparing the present invention (FIG. 6 and FIG. 7) and the conventional method (FIG. 1), the amount of diluting water used is 1,950,000 tons per year in total in the conventional method (FIG. 1) whereas the amount of diluting water used is 1,100,000 tons per year in total in the present invention (FIG. 6 and FIG. 7).

In addition, the ethanol concentration in the obtained fermentation broth is 7.3 mass % in the conventional method, whereas this is improved to 10 mass % in the present invention. That is, in the present invention, it is possible to reduce the necessary energy required for distillation when obtaining the ethanol product (ethanol with a purity of about 100%).

According to the method of producing ethanol of the present invention, it is possible to reduce the amount of diluting water to be added and to increase the ethanol concentration in the obtained fermentation broth.

Here, in the method of producing ethanol of the present invention, using the “sugar concentration measurement means” and the “ethanol concentration and sugar concentration measurement means” as shown in FIG. 6 and FIG. 7 respectively, the measurement of the sugar concentration in the sugar solution derived from the cellulosic raw materials or in the sugar-containing liquid and the measurement of the sugar and ethanol concentrations in the fermentation broth derived from the cellulosic raw materials are performed. Thereafter, the amount of sugar-containing liquid, cellulosic raw material treatment liquid or the amount of the diluting water is preferably determined according to the control concept or formula shown in FIG. 2 to FIG. 5 using the obtained measurement values or the like.

In addition, in FIG. 7, the ethanol concentration in the fermentation broth derived from cellulosic raw materials is measured using the “ethanol concentration and sugar concentration measurement means” and the amount of fermentation broth derived from the cellulosic raw materials which is added to the sugar-containing liquid is determined. However, in a case where there is a surplus of the fermentation broth derived from cellulosic raw materials in the producing line because the ethanol concentration in the fermentation broth derived from cellulosic raw materials is high, for example, it is possible to perform direct distillation without adding the fermentation broth to the sugar-containing liquid.

### EXAMPLES

Although the present invention is described in further detail by showing the following examples, the present invention is not limited to the following examples.

### Examples 1 and 2, Comparative Examples 1 and 2

The ethanol concentration in the fermentation broth after fermentation was investigated in a case where the ethanol was produced using bagasse as the cellulosic raw material and molasses as the sugar-containing liquid.

### Test Example 1 Preparation of Yeast Propagate Solution

After the sterilization of a medium including yeast extract 10 g/L, peptone 20 g/L, and glucose 20 g/L using a conventional method, this strain was inoculated and propagated aerobically for 1 day at 35° C. The propagated solution was used as is in the molasses fermentation and the bagasse fermentation.

### Test Example 2 Preparation of Enzyme Solution

After the sterilization of a medium (pH 4.0) including crystalline cellulose 50 g/L, corn steep liquor 10 g/L, ammonium sulfate 5 g/L, urea 3 g/L, magnesium sulfate 1.2 g/L, potassium dihydrogen phosphate 12 g/L, zinc sulfate 10 mg/L, manganese sulfate 10 mg/L, and copper sulfate 10 mg/L, this bacterial strain was inoculated and propagated aerobically for 7 days at 30° C. The obtained propagated solution was used as is in the bagasse saccharification.

### Example 1

### Bagasse Saccharification

As the pre-treatment of the enzymatic saccharification, a hydrothermal treatment of bagasse, which is a cellulosic raw material, was performed. In the hydrothermal treatment, a small pressure vessel (steam gun) with a biomass input port, a reactant discharge port, and a steam supply port was used. 100 g of bagasse (water content 20 mass %) was introduced and sealed into the steam gun and heated to 220° C. by supplying steam. After being held in this state for 10 minutes, the discharge port was opened and the bagasse was taken out. The total amount of discharged matter was 140 g, and the water content was 60 mass %.

The above-mentioned treated bagasse was used as is in the enzymatic saccharification or the simultaneous saccharification and fermentation.

25 g of the hydrothermally-treated bagasse (wet base, water content 60 mass %) was placed into a 250 mL sterilized conical flask, 6.7 g of an enzyme liquid and 18.3 g of sterilized water were added, and a slurry with 20 mass % of hydrothermal bagasse was prepared (total amount 50 g). The flask was shaken for 96 hours at 50 rpm in a shaking incubator (NX-25D produced by Nissin Rika Ltd. Shaking devices cited below are the same model) provided in a 35° C. constant temperature incubator, and saccharification was performed. The total amount after the saccharification was 50 g.

### (Molasses Fermentation)

18 g of molasses (sugar concentration 50 mass %) was placed into a sterilized conical flask and 4.4 g of the yeast propagation solution of Test Example 1, 50 g of the above-described bagasse saccharification liquid, and 16.4 g of sterilized water including 0.5 g of ammonium sulfate was added thereto to make the total amount 88.8 g. The flask was shaken for 48 hours at 50 rpm in a shaking incubator installed in a 35° C. constant temperature incubator, and fermentation was performed. The total amount after the fermentation was 80.7 g and the ethanol concentration was 10 mass %.

### Example 2

### Simultaneous Saccharification and Fermentation of Bagasse

25 g of the hydrothermally-treated bagasse (wet base, water content 60 mass %) was placed into a 250 mL sterilized conical flask in the same manner as in Example 1, 2.5 g of yeast propagate solution, 6.7 g of an enzyme liquid and 15.8 g of sterilized water were added, and a slurry with 20 mass % of hydrothermal bagasse was prepared (total amount 50 g). The flask was shaken for 96 hours at 50 rpm in a shaking incubator installed in a 35° C. constant temperature incubator, and simultaneous saccharification and fermentation were performed. The total amount after the simultaneous saccharification and fermentation was 46.4 g and the ethanol concentration was 7.7 mass %.

### (Molasses Fermentation)

18 g of molasses (sugar concentration 50 mass %) was placed into a sterilized conical flask and 4.3 g of a yeast propagate solution, 46.4 g of the above-described bagasse fermentation broth, and 16.5 g of sterilized water including 0.5 g of ammonium sulfate was added thereto to make the total amount 85.2 g. The flask was shaken for 48 hours at 50 rpm in a shaking incubator installed in a 35° C. constant temperature incubator, and fermentation was performed. The total amount after the fermentation was 80.7 g and the ethanol concentration thereof was 10 mass %.

### Comparative Example 1

### Simultaneous Saccharification and Fermentation of Bagasse

25 g of the hydrothermally-treated bagasse (wet base, water content 60 mass %) was placed into a 250 mL sterilized conical flask in the same manner as in Example 1, 2.5 g of the yeast propagate solution of Test Example 1, 6.7 g of the enzyme liquid of Test Example 2 and 15.8 g of sterilized water were added, and a slurry with 20 mass % of hydrothermal treated bagasse was prepared (total amount 50 g). The flask was shaken for 96 hours at 50 rpm in a shaking incubator installed in a 35° C. constant temperature incubator, and simultaneous saccharification and fermentation were performed. The total amount of the fermentation broth after the simultaneous saccharification and fermentation was 46.4 g and the ethanol concentration of the fermentation broth was 7.7 mass %.

### (Molasses Fermentation)

18 g of molasses (sugar concentration 50 mass %) was placed into a sterilized conical flask and 2.5 g of the yeast propagate solution of Test Example 1, and 29.5 g of sterilized water including 0.5 g of ammonium sulfate was added thereto to make the total amount 50 g. The flask was shaken for 48 hours at 50 rpm in a shaking incubator installed in a 35° C. constant temperature incubator, and fermentation was performed. The total amount of fermentation broth after the fermentation was 45.5 g and the ethanol concentration of the fermentation broth was 10 mass %.

The amounts and concentrations of the ethanol where the two fermentation broths are combined are shown in Table 1.

### Comparative Example 2

### Simultaneous Saccharification and Fermentation of Bagasse

25 g of the hydrothermally-treated bagasse (wet base, water content 60 mass %) was placed into a 250 mL sterilized conical flask in the same manner as in Example 1, 2.2 g of the yeast propagate solution of Test Example 1, 6.7 g of the enzyme liquid of Test Example 2 and 10.8 g of sterilized water were added, and a slurry with 22.4 mass % of hydrothermal bagasse was prepared (total amount 44.7 g). The flask was shaken for 96 hours at 50 rpm in a shaking incubator installed in a 35° C. constant temperature incubator, and simultaneous saccharification and fermentation were performed. The total amount after the simultaneous saccharification and fermentation was 41.7 g and the ethanol concentration was 7.3 mass %.

### (Molasses Fermentation)

18 g of molasses (sugar concentration 50 mass %) was placed into a sterilized conical flask and 2.2 g of the yeast propagate solution of Test Example 1, and 24.5 g of sterilized water including 0.5 g of ammonium sulfate was added thereto to make the total amount 44.7 g. The flask was shaken for 48 hours at 50 rpm in a shaking incubator installed in a 35° C. constant temperature incubator, and fermentation was performed. The total amount after the fermentation was 40.7 g, and the ethanol concentration thereof was 10 mass %.

The amounts and concentrations of the ethanol where the two fermentation broths are combined are shown in Table 1.

TABLE 1

| | | | Comparative | Comparative |
|---|---|---|---|---|
| | Example 1 | Example 2 | Example 1 | Example 2 |
| Total liquid | 80.7 g | 80.7 g | 91.1 g | 82.4 g |
| amount | | | | |
| Total ethanol | 8.1 g | 8.1 g | 8.1 g | 7.1 g |
| amount | | | | |
| Ethanol | 10.0 mass % | 10.0 mass % | 8.8 mass % | 8.6 mass % |
| concentration | | | | |

First, Comparative Example 1 is an example where bagasse and molasses are separately fermented. For both fermentation broths, conditions where the fermentations successfully proceed (bagasse saccharification and fermentation: slurry concentration of 20 mass % or less, ethanol concentration after fermentation of 10 mass % or less, and molasses fermentation: ethanol concentration after fermentation of 10 mass % or less) were adopted. It is considered that the maximum ethanol yield was obtained in consideration of the amounts of the molasses and the bagasse used.

Next, in the same manner as in Comparative Example 1, Comparative Example 2 is an example where the diluting water amount is reduced by 5 g in both steps with the object of improving the ethanol concentration in the treatment of fermenting the bagasse and the molasses separately. In such a case, the ethanol yields of both systems were reduced. Furthermore, in addition to the ethanol yield being low as a result, the ethanol concentration was also lowered.

On the other hand, Examples 1 and 2 according to the present invention are examples where the fermentation broth derived from the sugar-containing liquid was used in place of the sterilized water as the diluting water while ensuring conditions where the above-described fermentations proceed well. As a result, the ethanol yield obtained was greater than the Comparative Example 2 and the ethanol concentration in the obtained fermentation broth was higher than the Comparative Example 1.

From the above-described results, according to the method of producing ethanol of the present invention, it is understood that it is possible to produce high concentration ethanol with high efficiency from the cellulosic raw materials even in a case where the same amounts of molasses and bagasse are used.

### Reference Example 1

The relationship between the ethanol concentration in the fermentation broth and the amount of required heat for the distillation of ethanol in the fermentation broth was investigated by performing simulations.

The amount of energy required during the distillation of the fermentation broth and the ethanol concentration in the fermentation broth are in an inversely proportional relationship. Accordingly, the amount of energy required in a case where approximately 5 mass % of ethanol is concentrated and dehydrated into 99.5 mass % is approximately 1390 kcal/L-ethanol and the amount of energy required in a case where approximately 8 mass % of ethanol is concentrated and dehydrated into 99.5 mass % is approximately 930 kcal/L-ethanol.

From the results of the above-described simulations, it is understood that the higher the ethanol concentration in the fermentation broth obtained by the fermenting step, the more it is possible to reduce the amount of heat required for the distillation after the ferment step.

### INDUSTRIAL APPLICABILITY

Since it is possible to produce high concentration ethanol with high efficiency from cellulosic raw materials and sugar-containing liquid by using the method of producing ethanol of the present invention, it is possible to be favorably used in the field of ethanol production from biomass.

1. A method of producing ethanol by adding a cellulosic raw material treatment liquid to a sugar-containing liquid and then performing ethanol fermentation,
wherein the sugar-containing liquid is one or more types selected from the group consisting of extracted juice of crops, molasses, and enzyme-treated product of cereals containing a water-soluble saccharide, and
the cellulosic raw material treatment liquid is either a sugar solution derived from cellulosic raw materials obtained by saccharification of the cellulosic raw materials or a fermentation broth derived from the cellulosic raw materials obtained by ethanol fermentation of the sugar solution derived from the cellulosic raw materials.

2. The method of producing ethanol according to claim 1, wherein the sugar concentration in a fermentation tank for ethanol fermentation of the sugar-containing liquid is 25 mass % or less.

3. The method of producing ethanol according to claim 1, wherein the ethanol concentration obtained after the ethanol fermentation of the sugar-containing liquid is 12 mass % or less.

4. The method of producing ethanol according to claim 1, wherein a saccharifying step and a fermenting step of the cellulosic raw material are performed in the same reaction tank.

5. The method of producing ethanol according to claim 1, wherein the cellulosic raw material is one or more types selected from the group consisting of bagasse, rice straw, wheat straw, chaff, wheat shells, cassava residue, and corn stover, and the enzyme-treated product of the cereal is a starch saccharification liquid obtained by saccharification of one or more types selected from the group consisting of rice, wheat, cassava, and corn.

6. The method of producing ethanol according to claim 1, wherein the cellulosic raw material is bagasse and the sugar-containing liquid is extracted juice of crops containing a water-soluble saccharide or molasses.

7. The method of producing ethanol according to claim 2, wherein the ethanol concentration obtained after the ethanol fermentation of the sugar-containing liquid is 12 mass % or less.

8. The method of producing ethanol according to claim 2, wherein a saccharifying step and a fermenting step of the cellulosic raw material are performed in the same reaction tank.

9. The method of producing ethanol according to claim 2, wherein the cellulosic raw material is one or more types selected from the group consisting of bagasse, rice straw, wheat straw, chaff, wheat shells, cassava residue, and corn stover, and the enzyme-treated product of the cereal is a starch saccharification liquid obtained by saccharification of one or more types selected from the group consisting of rice, wheat, cassava, and corn.

10. The method of producing ethanol according to claim 2, wherein the cellulosic raw material is bagasse and the sugar-containing liquid is extracted juice of crops containing a water-soluble saccharide or molasses.

11. The method of producing ethanol according to claim 3, wherein a saccharifying step and a fermenting step of the cellulosic raw material are performed in the same reaction tank.

12. The method of producing ethanol according to claim 3, wherein the cellulosic raw material is one or more types selected from the group consisting of bagasse, rice straw, wheat straw, chaff, wheat shells, cassava residue, and corn stover, and the enzyme-treated product of the cereal is a starch saccharification liquid obtained by saccharification of one or more types selected from the group consisting of rice, wheat, cassava, and corn.

13. The method of producing ethanol according to claim 3, wherein the cellulosic raw material is bagasse and the sugar-containing liquid is extracted juice of crops containing a water-soluble saccharide or molasses.

14. The method of producing ethanol according to claim 4, wherein the cellulosic raw material is one or more types selected from the group consisting of bagasse, rice straw, wheat straw, chaff, wheat shells, cassava residue, and corn stover, and the enzyme-treated product of the cereal is a starch saccharification liquid obtained by saccharification of one or more types selected from the group consisting of rice, wheat, cassava, and corn.

15. The method of producing ethanol according to claim 4, wherein the cellulosic raw material is bagasse and the sugar-containing liquid is extracted juice of crops containing a water-soluble saccharide or molasses.

16. The method of producing ethanol according to claim 5, wherein the cellulosic raw material is bagasse and the sugar-containing liquid is extracted juice of crops containing a water-soluble saccharide or molasses.
